(19) United States
(12) Patent Application Publication
(10) Pub. No.: US 2018/0364260 A1
(43) Pub. Date: Dec. 20, 2018

(54) METHODS FOR EXTRACTING AND MEASURING CONCENTRATIONS OF BIOMOLECULES IN COMPLEX MATRICES WITHOUT THE NEED FOR IMMUNOCAPTURE

(71) Applicant: C2N DIAGNOSTICS LLC, SAINT LOUIS, MO (US)

(72) Inventor: Matthew Richard Meyer, Chesterfield, MO (US)

(21) Appl. No.: 15/981,767

(22) Filed: May 16, 2018

Related U.S. Application Data
(60) Provisional application No. 62/521,249, filed on Jun. 16, 2017.

(51) Int. Cl. G01N 33/68; G01N 33/96

(52) U.S. Cl. CPC G01N 33/6896; G01N 2800/2821; G01N 33/96

(57) ABSTRACT

Extraction and separation methods that can be used to quantify the absolute concentrations of one or more low abundance biomolecules present in biological fluids and tissues, without the need for antibody enrichment or reliance on an antibody for quantification are provided. These biomolecules can be biomarkers used to diagnose and monitor disease progression. As an example, these methods are applied to human plasma and CSF, and used to quantify Alzheimer's disease biomarkers.

### CROSS-REFERENCE TO RELATED APPLICATIONS

This application claims benefit of priority under 35 U.S.C. § 119(e) of U.S. Ser. No. 62/521,249, filed May 16, 2017, the entire contents of which is incorporated herein by reference in its entirety.

### INCORPORATION OF SEQUENCE LISTING

The material in the accompanying sequence listing is hereby incorporated by reference into this application. The accompanying sequence listing text file, name C2N1150_1_Sequence_Listing.txt, was created on May 8, 2018, and is 10 kb. The file can be assessed using Microsoft Word on a computer that uses Windows OS.

### FIELD OF THE INVENTION

The invention generally relates to methods for the extraction of biomolecules from complex matrices and the utility for detecting, identifying, characterizing, and quantifying these biomolecules.

### BACKGROUND OF THE INVENTION

Challenge of Extracting and Separating Biomolecules.

A need exists for sensitive, accurate, and reproducible methods for extracting and quantifying biomolecules present in human and animal biofluids and tissues. Several extraction approaches exist that enrich for broad biomolecular classes (e.g., proteins/peptides, lipids, carbohydrates, nucleic acids). These include, but are not limited to, immunocapture, acid precipitation, hydrolysis, solid phase extraction, liquid-liquid extraction, chromatography, electrophoresis, and centrifugation. However, no one approach is ideally selective for a single biomolecular class. So, each extraction approach may enrich for a select subset of biomolecules, but it is well known that an extract targeted to one biomolecular class will also contain some compounds from other biomolecular classes. These are often referred to as contaminants that can confound downstream detection and analytical methods. However, in some cases these ‘contaminants’ might represent biomolecular targets that are impractical to extract and detect using other available enrichment strategies.

Antibodies: Limitations and Strengths for Extracting and Separating Proteins/Peptides.

In particular, proteins and peptides are heterogeneous and possess diverse physicochemical properties, so that a single extraction approach will not enrich for all proteins/peptides of interest that are present in a biological sample. The use of antibodies directed at a particular protein/peptide significantly enhances selectivity for protein/peptide extraction approaches (immunoprecipitation or immunocapture), but are still limited by several parameters, including commercial availability, species specificity, epitope specificity vs. cross-reactivity, affinity and avidity for the targeted epitope, protein/peptide structure that can limit antibody access to the targeted epitope, alterations in protein/peptide composition (chemical modifications to amino acid residues), and the presence of protein variants or isoforms that differ only slightly from the targeted epitope and will not be captured (immunoprecipitated) by the antibody. These issues can be especially limiting in situations where the protein or peptide of interest is of very low abundance and the biofluid contains a complex mixture of other proteins, lipids, and biomolecules. Additionally, optimizing the time and incubation conditions required to most efficiently capture or precipitate the protein/peptide target from the biofluid further limits the use of antibodies for protein/peptide extraction and subsequent detection, identification, characterization, and quantification.

Alternative Approaches.

One potential alternative for targeted protein/peptide extraction is to identify and exploit physicochemical properties common among the proteins/peptides of interest rather than the affinity of an antibody against the protein/peptide. The properties include, but are not limited to, protein/peptide size, mass, charge state, amino acid sequence and composition, post-translational modifications, structure/folding, freezing or melting temperature, and solubility in various aqueous or organic solvents. The latter approach is particularly relevant in this application.

Others have exploited aqueous vs. organic solubility for extracting and separating biomolecules. For example, proteins/peptides are generally soluble in aqueous solutions and can be extracted and separated from lipids that are generally soluble in organic solutions, while biomolecules that are insoluble in aqueous and organic solvents precipitate. Substantial efficiency is realized when these solvents are used in a single sample tube/vial where the aqueous and organic solvent phases naturally separate, and centrifugation deposits the precipitate at the bottom of the tube. However, we have discovered that several hydrophobic proteins/peptides are extracted into the organic solvent and precipitate. These represent low abundance, biologically relevant proteins/peptides that have been enriched in the organic phase or pellet by separating them from the large number of more abundant proteins/peptides extracted into the aqueous phase.

What about the Lipids?

Clearly, the organic solvent fraction and precipitate will contain many abundant lipids whose presence can compromise downstream detection and analysis of other biomolecules, and especially any enriched proteins/peptides. However, herein we have implemented several liquid:liquid extraction approaches that separate the lipids from the biomolecules and proteins/peptides of interest. The proteins/peptides are further enriched by this delipidation step and are suitable for downstream analysis. Those skilled in the art will appreciate whether delipidation is necessary for their sample extraction and suitable for their selected analytical platform.

Plasma and Cerebrospinal Fluid (CSF) Protein Biomarkers for Alzheimer's Disease: An Example.

Alzheimer's disease (AD) is the most common cause of dementia and is an increasing public health problem. It is currently estimated to afflict 5 million people in the United States, with an expected increase to 13 million by the year 2050 (Herbert et al, 2001, Alzheimer Dis. Assoc. Disord. 15(4): 169-173). AD, like other central nervous system (CNS) degenerative diseases, is characterized by disturbances in protein production, accumulation, and clearance. In AD, dysregulation in the metabolism of the protein, amyloid-beta (Aβ), is indicated by a massive buildup of this protein as amyloid plaques formed in the brain of those with the disease. In addition the protein tau builds up in the brain in the form of tau tangles. AD leads to loss of memory, cognitive function, independence, and ultimately death. The disease takes a heavy personal and financial toll on the patient, the family, and society. Because of the severity and increasing prevalence of this disease in the population, it is urgent that better diagnostics and treatments be developed.

Currently, there are some medications that modify AD symptoms, however, there are no disease-modifying treatments. Disease-modifying treatments will likely be most effective when given before the onset of irreversible brain damage. However, by the time clinical diagnosis of AD is made, extensive neuronal loss has already occurred (Price et al. 2001, Arch. Neurol. 58(9): 1395-1402). Therefore, a plasma or CSF biomarker is needed to identify those at risk of developing AD, so that treatments can be administered that might prevent or delay AD onset. Currently, there are no established plasma protein/peptide biomarkers that clearly identify the pathophysiologic changes that occur in AD before the onset of clinical symptoms or that monitor the effects of treatments that may prevent the onset or slow the progression of the disease.

### SUMMARY OF THE INVENTION

Among the various aspects of the present invention is a method for extraction and separation to detect, identify, characterize, and quantify the absolute concentrations of one or more low abundance biomolecules present in biological fluids and tissues, without the need for antibody enrichment or reliance on an antibody for quantification. These biomolecules can be biomarkers used to diagnose and monitor disease progression in a subject. In one embodiment, the biomolecules are extracted from a complex background matrix by precipitation with an organic solvent, organic acid, base, or inorganic salt. In another embodiment, the biomolecules are extracted by liquid-liquid phase separation using an organic solvent to further separate the biomolecules from complex biological matrices, wherein the organic solvent could be, but not limited to, methyl tert-butyl ether, methanol, ethanol, isopropanol, chloroform, ethyl acetate, trichloroethylene, dichloromethane, xylenes, hexane, or the solvents outlined in the GMP Q3C Solvent Tables and List (FDA Guidance, ICH, 2012, revision 2). In another embodiment, those skilled in the art can appreciate that mixtures of two or more solvents could be used for biomolecule extraction. In yet another embodiment, an aqueous two-phase system (ATPS) could be utilized to extract biomolecules of interest. These extracted biomolecules can then be detected, identified, characterized, and quantified using any available device for biomolecule detection, including but not limited to, liquid chromatography, mass spectrometry, or a combination thereof.

In another aspect, this method can be used for calculating the concentration of one or more biomolecules in a subject. The method includes contacting a sample from the subject with a Quantitation Standard, where the Quantitation Standard is a known concentration of a labeled biomolecule of interest. The Quantitation Standard can be contacting the sample from the subject either before or after extraction of the biomolecules of interest from the sample. The method further includes extracting biomolecules of interest from the sample and determining a ratio for each biomolecule to its corresponding Quantitation Standard in the sample. The ratios are used to calculate the concentration of the unlabeled biomolecule in the sample. In one embodiment, the method further includes normalizing the calculated concentration to a standard curve, wherein the standard curve is generated by determining two or more ratios of unlabeled biomolecules to their corresponding Quantitation Standard, where the concentration of the unlabeled biomolecule is known. In another embodiment, the concentration of two unlabeled biomolecules can be measured in the same sample, wherein the ratio of one biomolecule to the other can be used to monitor or diagnose disease and/or disease progression in a subject.

In another aspect, the present invention provides an in vivo method for quantifying the concentration of one or more biomolecules in a subject. The method further includes obtaining a sample of biological fluid or tissue from the subject, where the sample includes an unlabeled biomolecule fraction. The sample is then contacted with a Quantitation Standard, where the Quantitation Standard includes a known concentration of a biomolecule labeled with a moiety that has a molecular weight that differs from the unlabeled biomolecule. In one embodiment, calculating the concentration of the unlabeled biomolecule comprises multiplying the concentration of the Quantitation Standard with the determined ratio of unlabeled biomolecule to the Quantitation Standard. In another embodiment, the calculated concentration of unlabeled biomolecule is normalized to a standard curve, wherein the standard curve is generated by determining two or more ratios of unlabeled biomolecules to Quantitation Standard, where the concentration of unlabeled biomolecule is known.

In another aspect, the invention provides a method for determining whether a therapeutic agent affects the concentration of a biomolecule produced in the central nervous system, peripheral tissues, or biofluids of a subject. The method comprises administering a therapeutic agent to the central nervous system, peripheral tissue, or biofluid of the subject. The method further comprises obtaining a biological sample from the subject, wherein the biological sample contains an unlabeled biomolecule. The next step of the process comprises detecting the amount of unlabeled biomolecule, wherein the ratio of unlabeled biomolecule to the Quantitation Standard can be used to determine the concentration of the biomolecule in the subject. The final step of the process comprises comparing the concentration of the biomolecule in the subject to a suitable control value (“cut-off” or “threshold value”), wherein a deviation from the control value indicates the therapeutic agent affects the concentration of the biomolecule in the central nervous system, peripheral tissues, or biofluids of the subject.

In some aspects, the labeled moiety used for the Quantitation Standard includes a non-radioactive isotope that is selected from the group consisting of ²H, ¹³C, ¹⁵N, ¹⁷O, ¹⁸O, ³³S, ³⁴S, ³⁶S, ⁷⁴Se, ⁷⁶Se, ⁷⁷Se, ⁷⁸Se, and ⁸⁰Se. In one embodiment, the labeled moiety is a labeled amino acid. Exemplary amino acids include, but are not limited to threonine, leucine, and isoleucine. Thus, in one embodiment, the labeled moiety is ¹³Cₓ-threonine, where x=1 to 4. In another embodiment, the labeled moiety is a ¹⁵N-labeled amino acid. In another embodiment, the labeled moiety is a ¹³Cₓ-labeled leucine, where x=1 to 6. In another embodiment, the labeled moiety is a ¹³Cₓ-labeled glutamic acid, where x=1 to 5. In another embodiment, the labeled moiety is a ¹³Cₓ-labeled glycine, where x=1 to 2. In another embodiment, the labeled moiety is a ¹³Cₓ-labeled alanine, where x=1 to 3. In another embodiment, the labeled moiety is a ¹³Cₓ-labeled isoleucine, where x=1 to 6. In another embodiment, wherein the labeled moiety is ¹³Cₓ-valine, where x=1 to 5. In another embodiment, wherein the labeled moiety is ¹³Cₓ-methionine, where x=1 to 5. In another embodiment, wherein the labeled moiety is ^{33, 34, 35}S-methionine. In another embodiment, wherein the labeled moiety is ^{74, 76, 77, 78, 80}Se-methionine. In another embodiment, the labeled moiety is a ¹³Cₓ-labeled lysine, where x=1 to 6

In all aspects, the biomolecule may be a protein/peptide, lipid, nucleic acid, metabolite, or carbohydrate. In one embodiment, the biomolecule is a peptide that is synthesized in the central nervous system (CNS) such as tau, amyloid-beta (Aβ), alpha-synuclein, apolipoprotein E, apolipoprotein J, amyloid precursor protein (APP), alpha-2-macroglobulin, S100B, myelin basic protein, TDP-43, superoxide dismutase-1, huntingtin, an interleukin, and tumor necrosis factor (TNF). In aspects of the invention where two or more biomolecules are detected, identified, characterized, and quantified, the biomolecules may be isoforms of the same protein. As such, in one embodiment, the biomolecule may be one or more of amyloid-β 1-36, amyloid-β 1-37, amyloid-β 1-38, amyloid-β 1-39, amyloid-β 1-40, amyloid-β 1-42, amyloid-β 1-43, amyloid-β 1-45, amyloid-β 1-46, and amyloid-β 1-48.

Other aspects and features of the invention are described in more detail below.

### BRIEF DESCRIPTION OF THE DRAWINGS

FIG. 1 depicts the digestion scheme of the Aβ isoforms with cyanogen bromide. This approach produces isoform specific Aβ C-terminal fragments (CTFs) that can be isolated by liquid-liquid extraction with an organic solvent. In one embodiment, differences in the mass-to-charge (m/z) of Aβ CTFs can be distinguished and quantified using liquid chromatography and mass spectrometry.

FIG. 2 exemplifies the processing workflow for Aβ CTFs enrichment and extraction from a biological sample (e.g., human plasma) obtained from a subject. Total plasma protein in the sample is precipitated and resolubilized in formic acid. The solubilized protein pellet is digested with cyanogen bromide or another endoprotease to yield isoform specific Aβ CTFs. The digested sample then undergoes liquid-liquid extraction with an organic solvent to extract the CTFs. In one embodiment, the CTFs are detected, identified, characterized, and quantified using liquid chromatography-mass spectrometry.

FIGS. 3A and 3B depict the calibration curves for the Aβ40 and Aβ42 CTFs generated in an artificial plasma matrix using the processing method described above. The curves show linearity across a dynamic range of three orders of magnitude. The calculated coefficients of variation (CVs) for each standard point were less than 20% for both the Aβ40 and Aβ42 calibration curves indicating good reproducibility of the LC-MS analysis (FIG. 3C). The estimated lower limits of quantification (CVs <20% and accuracy between 80-120%) for Aβ40 and Aβ42 are 195 pg/ml and 29 pg/ml, respectively (FIG. 3C and FIG. 5B).

FIG. 4 shows the extracted ion chromatograms for the most abundant fragment ions of the endogenous (‘unlabeled’; upper panel) and ¹⁵N labeled internal standard (lower panel) Aβ40 and Aβ42 C-terminal fragments extracted from 400 μl of pooled human plasma spiked with ¹⁵N-Aβ40 and ¹⁵N-Aβ42 internal standards. The calculated concentrations of endogenous Aβ40 and Aβ42 are 1220.1 pg/ml and 224.7 pg/ml, respectively, in this pool. This demonstrates that this process is suitable for extracting and quantifying human plasma Aβ isoforms without the need for immunoprecipitation.

FIGS. 5A and 5B. FIG. 5A depicts the reproducibility of Aβ40 and Aβ42 concentration measurements from a set of five QC plasma samples that were processed and analyzed on five separate days. The calculated concentrations of endogenous Aβ40 and Aβ42 for each QC are shown in FIG. 5B. The inter-assay CVs for repeat measurements of Aβ40 and Aβ42 concentrations and the Aβ42/Aβ40 concentration ratio are less than 15%. This data demonstrates the reproducibility of the extraction process and its utility for quantifying human plasma Aβ isoforms without the need for immunoprecipitation.

FIG. 6 depicts the digestion scheme of the Aβ isoforms with a palladium (Pd(II)) metal peptidase which cleaves between the two residues upstream (towards the N-terminal) from the coordinating methionine residue. In one embodiment, this approach produces isoform specific Aβ C-terminal fragments that can be detected, identified, characterized, and quantified using liquid chromatography and mass spectrometry.

FIG. 7 shows the extracted ion chromatograms for the most intense fragment ions from an equimolar mixture of ¹⁴N (upper panel) and ¹⁵N labeled (lower panel) Aβ38 and Aβ40 recombinant protein standards, generated by digestion with a Pd(II) metal peptidase. The Aβ CTFs were extracted from the digestion reaction with an organic solvent. This demonstrates the versatility of this process as it is capable of isolating and quantifying a different subset of Aβ isoform-specific CTFs.

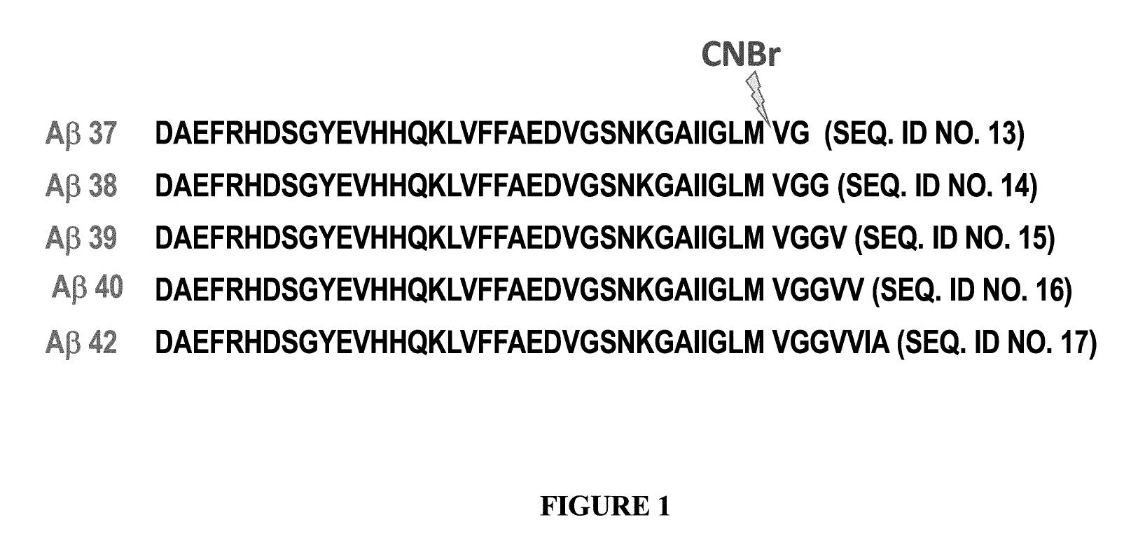

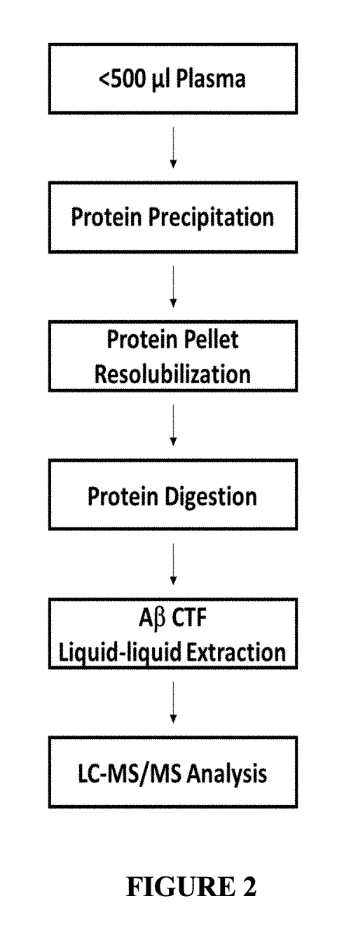

### DETAILED DESCRIPTION OF THE INVENTION

The present invention is based, in part, on the discovery that the presence of stable isotope labeled atoms in biomolecules leads to small predictable differences in molecular weight of the biomolecules, but does not alter the physical or chemical properties of the biomolecules. Using the techniques provided herein, quantitative analysis of biomolecules can be used to diagnose and/or treat a subject having or at risk of developing a neurological, neurodegenerative, or pathophysiological disorder. Accordingly, the present invention provides methods for detecting and calculating the concentration of one or more biomolecules of interest in a biofluid obtained from a subject without the need for immunoprecipitation of the target biomolecule with an antibody.

The invention also provides a method to assess whether a therapeutic agent affects the abundance of biomolecules in the subject, where the biomolecules are relevant to neurological, neurodegenerative, or pathophysiological diseases. Accordingly, the method may be used to determine the optimal doses and/or optimal dosing regimen of the therapeutic agent. Additionally, the method may be used to determine target engagement of a particular therapeutic agent with the biomolecule. Alternatively, subjects with one particular genotype may respond better to a particular therapeutic agent than those with a different genotype. Finally, by allowing isoform specific quantitation, the method may be used to determine whether a therapeutic agent modulates the abundance of a biomolecule by switching production of one isoform to another isoform of the same biomolecule.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. In addition, the term “subject” may refer to a culture of cells or the solution surrounding the cells (“culture media”), where the methods of the invention are performed in vitro to assess, for example, efficacy of a therapeutic agent, or cellular production or secretion of biomolecules of interest.

As used herein, the terms “sample” and “biological sample” refer to any sample suitable for the methods provided by the present invention. A sample of cells used in the present method can be obtained from tissue samples or bodily fluid from a subject, or tissue obtained by a biopsy procedure (e.g., a needle biopsy) or a surgical procedure. In certain embodiments, the biological sample of the present invention is a sample of bodily fluid, e.g., cerebral spinal fluid (CSF), blood, plasma, serum, blood cells, urine, saliva, vitreous fluid, perspiration (“sweat’), breath condensate, and tears.

As disclosed herein, stable isotope labeling of biomolecules leads to small differences in molecular weight of the biomolecules, but does not alter the physical or chemical properties of the biomolecules. In one embodiment, the Quantitation Standards can be composed of stable isotopes, and these labeled biomolecules will behave similarly to endogenous (unlabeled) biomolecules during sample preparation and coelute on a liquid chromatography column in an identical fashion. Sensitive instruments, such as mass spectrometers, provide the ability to measure small differences in mass between labeled and unlabeled biomolecules.

Accordingly, in one aspect, the invention provides a method of calculating the concentration of a biomolecule in a subject. In one embodiment, the method includes contacting a sample from the subject with a Quantitation Standard. In one embodiment and disclosed herein, a “Quantitation Standard” refers to a known concentration of a labeled biomolecule, which has a distinct molecular weight from other labeled or unlabeled biomolecules that may exist in the sample. Thereafter, a sensitive measuring device, such as a mass spectrometer, a tandem mass spectrometer, or a combination of both, is used to quantify the signal intensities of labeled to unlabeled biomolecules and express them as a ratio. Because the physical properties of the labeled and unlabeled biomolecules are identical, the ratio measured by the mass spectrometer is identical to the ratio in the original sample. Thus, by adding a known amount of one or more biomolecules, each labeled with a unique isotope composition or pattern, the invention provides the ability to quantitate the amount of those biomolecules that have different isotopic composition.

As used herein, the term “biomolecule” refers to any organic molecule in a living organism. Exemplary biomolecules include, but are not limited to, proteins/peptides, lipids, nucleic acids, metabolites, and carbohydrates. In one embodiment, the biomolecule is a peptide, such as a protein, that is synthesized in the central nervous system (CNS) of the subject. Exemplary proteins that can be measured by the methods described in this invention include, but are not limited to, tau (associated with Alzheimer's Disease), amyloid-β (Aβ) and its variants, soluble amyloid precursor protein (APP), apolipoprotein E (isoforms 2, 3, or 4), apolipoprotein J (also called clusterin), phosphorylated tau, glial fibrillary acidic protein, alpha-2 macroglobulin, alpha-synuclein, S100B, myelin basic protein (implicated in multiple sclerosis), prions, interleukins, TDP-43, superoxide dismutase-1, huntingtin, tumor necrosis factor (TNF), heat shock protein 90 (HSP90), and combinations thereof. Additional biomolecules that may be targeted include products of, or proteins or peptides that interact with, GABAergic neurons, noradrenergic neurons, histaminergic neurons, serotonergic neurons, dopaminergic neurons, cholinergic neurons, glutaminergic neurons, and those described in Table 2. In one embodiment, the protein whose in vivo concentration is measured may be an apolipoprotein E protein. In another embodiment, the protein whose in vivo concentration is measured may be alpha-synuclein. In another embodiment, the protein whose in vivo concentration is measured may be tau or its variants or isoforms. In another embodiment, the protein whose in vivo concentration is measured may be Aβ or its variants or isoforms. Exemplary isoforms of Aβ whose concentrations may be measured include, but are not limited to, the following phosphorylated, unphosphorylated, glycosylated, acetylated, nitrosylated, sulfated, methylated, amidated, deamidated, and oxidized isoforms of Aβ: amyloid-β 1-36, amyloid-β 1-37, amyloid-β 1-38, amyloid-β 1-39, amyloid-β 1-40, amyloid-β 1-42, and amyloid-β 1-43. The following shows a multiple sequence alignment of 7 different isoforms of Aβ.

Aβ1-36 DAEFRHDSGYEITHEIQKLVFFAEDVGSNKGAIIGLMV-----------(SEQ. ID No. 1)
Aβ1-37 DAEFRHDSGYEITHEIQKLVFFAEDVGSNKGAIIGLMVG----------(SEQ. ID No. 2)
Aβ1-38 DAEFRHDSGYEITHEIQKLVFFAEDVGSNKGAIIGLMVGG---------(SEQ. ID No. 3)
Aβ1-39 DAEFRHDSGYEITHEIQKLVFFAEDVGSNKGAIIGLMVGGV--------(SEQ. ID No. 4)
Aβ1-40 DAEFRHDSGYEITHEIQKLVFFAEDVGSNKGAIIGLMVGGVV-------(SEQ. ID No. 5)
Aβ1-41 DAEFRHDSGYEITHEIQKLVFFAEDVGSNKGAIIGLMVGGVVI------(SEQ. ID No. 6)
Aβ1-42 DAEFRHDSGYEITHEIQKLVFFAEDVGSNKGAIIGLMVGGVVIA-----(SEQ. ID No. 7)
Aβ1-43 DAEFRHDSGYEITHEIQKLVFFAEDVGSNKGAIIGLMVGGVVIAT----(SEQ. ID No. 8)

By way of example and not limitation, it is noted that several unique isoforms of Aβ exist in CSF and other biofluids, and that these isoforms can be post-translationally modified in several ways including phosphorylation, glycosylation, oxidation, nitration, methylation, and pyroglutamination. Cyanogen bromide (CNBr) digestion of Aβ yields several peptides which may or may not be unique to each isoform, see examples for Aβ40 and Aβ42 in Table 1. Thus, quantitation of some of these peptides allows for calculation of the concentration of these isoforms in the original biological fluid.

TABLE 1
CNBr digested peptides of Aβ40 and Aβ42
identified by LC-MS.

| | Peptide | Precursor | Isoform |
|---|---|---|---|
| | sequence | m/z | Specificity |
| SEQ. ID No. 9 | VGGVV | 430.2660 | Aβ40 |
| SEQ. ID No. 10 | ¹⁵N-VGGVV | 435.2512 | Aβ40 |
| SEQ. ID No. 11 | VGGVVIA | 614.3872 | Aβ42 |
| SEQ. ID No. 12 | ¹⁵N-VGGVVIA | 621.3664 | Aβ42 |

As such, the methods provide the ability to measure concentrations of various isoforms of Aβ, such as fragments produced after chemical digestion (e.g., CNBr, hydroxylamine, acids, BNPS-skatole, 2-nitro-5-thiocyanobenzoic acid, o-iodosobenzoic acid, or metal peptidases), digestion with an endoprotease (e.g., trypsin, Lys-N, Glu-C, LysArgiNase, Neprolysin, insulin degrading enzyme, MMP2, MMP9, MMP14, cathepsins, plasmin, or V8 protease), or photolysis. Exemplary fragments of Aβ isoforms include, but are not limited to the C-terminal regions of Aβ that are different among the different isoforms.

As used herein, the term “nucleic acid” refers to DNA, RNA, single-stranded, double-stranded or triple stranded and any chemical modifications thereof. Virtually any modification of the nucleic acid is contemplated. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule.

The terms “polypeptide,” “peptide” and “protein” are used interchangeably herein to refer to two or more amino acid residues joined to each other by peptide bonds or modified peptide bonds, i.e., peptide isosteres. The terms apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. “Polypeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration.

Several different moieties may be used to label the Quantitation Standard used for measuring a biomolecule of interest. Generally speaking, the two types of labeling moieties utilized in the method of the invention are radioactive isotopes and non-radioactive (stable) isotopes. In one embodiment, non-radioactive isotopes may be used and measured by mass spectrometry. Preferred stable isotopes include deuterium (²H), ¹³C ¹⁵N, ^{17 or 18}O, and ^{33, 34, or 36}S, ^{74, 76, 77, 78, or 80}Se, but it is recognized that a number of other stable isotopes that change the mass of an atom by more or less neutrons than is seen in the prevalent native form would also be effective. A suitable label generally will change the mass of the Quantitation Standard under study such that it can be detected in a mass spectrometer and distinguished from the endogenous biomolecule by a difference in mass-to-charge. In one embodiment, the Quantitation Standard utilized for quantification may be a peptide or protein, and the labeled moiety may be an amino acid comprising a non-radioactive isotope (e.g., ¹³C). In another embodiment, the Quantitation Standard may be a nucleic acid, and the labeled moiety may be a nucleoside triphosphate comprising a non-radioactive isotope (e.g., ¹⁵N). Alternatively, a radioactive isotope may be used, and the labeled Quantitation Standard may be measured with a scintillation counter (or via nuclear scintigraphy) as well as by a mass spectrometer. One or more labeled moieties may be used simultaneously or in sequence. In another embodiment, the Quantitation Standard may be labeled with a chemical moiety that fluoresces (e.g., green fluorescent protein) when exposed to energy (e.g. light) of a proper wavelength. In another embodiment, the Quantitation Standard may be labeled with an affinity tag (e.g., chitin binding protein (CBP), maltose binding protein (MBP), glutathione-S-transferase (GST), and poly-His). In another embodiment, the Quantitation Standard may be labeled with a solubilization tag (e.g., thioredoxin (TRX) and poly(NANP)). In another embodiment, the Quantitation Standard may be labeled with a chromatography tag (e.g., polyanionic amino acids, FLAG-tag). In another embodiment, the Quantitation Standard may be labeled with an epitope tag (e.g., V5-tag, Myc-tag, HA-tag and NE-tag). Those skilled in the art of biomolecule extraction will select appropriate Quantitation Standards based on the properties of the instrument chosen to detect, identify, characterize and quantify their biomolecules of interest.

Thus, in one embodiment, when the method is employed to measure the concentration of proteins, the labeled moiety of the Quantitation Standard typically will be an amino acid. Those of skill in the art will appreciate that several amino acids may be used to provide the labeled biomolecules. Generally, the choice of amino acid is based on a variety of factors such as: (1) the amino acid generally is present in at least one residue of the protein or peptide of interest. And (2) commercial availability of the desired amino acid (i.e., some amino acids are much more expensive or harder to manufacture than others).

There are numerous commercial sources of labeled amino acids, both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made with known synthetic chemical processes.

In one embodiment, longitudinal analysis of samples obtained from the subject given a pharmacologic agent (drug) would provide a measurement of concentrations of biomolecules of interest before and after drug administration, directly measuring the pharmacodynamic effect of the drug in the same subject.

Thus, once disease is established and a treatment protocol is initiated, the methods of the invention may be repeated on a regular basis to monitor the concentration(s) of biomolecule(s) of interest in the subject. The results obtained from successive assays may be used to show the efficacy of treatment over a period ranging from several days to months. Accordingly, another aspect of the invention is directed to methods for monitoring a therapeutic regimen for treating a subject having a neurological, neurodegenerative, or pathophysiological disorder. A comparison of the concentration(s) of biomolecule(s) of interest prior to and during therapy will be indicative of the efficacy of the therapy. Therefore, one skilled in the art will be able to recognize and adjust the therapeutic approach based on serial measurements of biomolecule concentrations.

The method of the invention provides that a sample be obtained from the subject such that the in vivo concentration of one or more biomolecules of interest can be determined. In one embodiment, the sample is a body fluid. Suitable body fluids include, but are not limited to, cerebral spinal fluid (CSF), blood plasma, blood serum, blood cells, urine, saliva, perspiration, vitreous fluid, breath condensate, and tears. It should be understood that biological fluids and cells typically contain a multitude of quantifiable biomolecules. For example, where the sample is plasma, exemplary biomolecules that can be quantified include, but are not limited to, tau, variants of tau, amyloid-beta protein, variants of amyloid-beta protein (Aβ), digestion products of amyloid-beta protein, amyloid precursor protein (APP), apolipoprotein E, apolipoprotein J, alpha-synuclein, the proteins defined in Table 2, or any combination thereof. In another embodiment, the sample is a tissue sample, such as a sample of tissue from the central nervous system (CNS). The sample generally will be collected using standard procedures well known to those of skill in the art.

In one embodiment, the sample is a CNS sample, which includes, but is not limited to, tissue from the central nervous system, which comprises brain tissue and spinal cord tissue. In one embodiment of the invention, the CNS sample may be taken from brain tissue, including, but not limited to, tissue from the forebrain (e.g., cerebral cortex, basal ganglia, hippocampus), the interbrain (e.g., thalamus, hypothalamus, subthalamus), the midbrain (e.g., tectum, tegmentum), or the hindbrain (e.g., pons, cerebellum, medulla oblongata). In another embodiment, the CNS sample may be collected from spinal cord tissue. In still other embodiments, CNS samples from more than one CNS region may be taken. Accordingly, the concentration of a biomolecule of interest may be measured in different CNS samples, e.g., in the cortex and the hippocampus, simultaneously.

CNS samples may be obtained by known techniques. For instance, brain tissue or spinal cord tissue may be obtained via dissection or resection. Alternatively, CNS samples may be obtained using laser microdissection. The subject may or may not have to be sacrificed to obtain the sample, depending on the CNS sample desired and the subject utilized.

In one embodiment, the sample is a blood sample, which includes, but is not limited to, tissue from the central nervous system; which comprises brain tissue and spinal cord tissue, tissue from peripheral organs, and muscle tissue. In other embodiments, blood samples from more than one region of the body may be taken. Accordingly, the concentrations of biomolecules of interest may be measured in different samples.

Blood samples may be obtained by known techniques. For instance, blood tissue may be obtained via venipuncture, finger stick, or heel stick. Alternatively, samples may be obtained using laser microdissection. The subject may or may not have to be sacrificed to obtain the sample, depending on the sample desired and the subject utilized.

Accordingly, the present invention provides that detection of the amount of unlabeled biomolecule and the amount of Quantitation Standard in the sample may be used to determine the ratio of unlabeled biomolecule to the Quantitation Standard, which in turn, may be used to calculate the concentration of the biomolecule of interest in the subject. Exemplary means for detecting differences in mass between the unlabeled biomolecules and the Quantitation Standards include, but are not limited to, liquid chromatography mass spectrometry, gas chromatography mass spectrometry, MALDI-TOF mass spectrometry, liquid chromatography MALDI-TOF mass spectrometry, and tandem mass spectrometry. In addition, one skilled in the art could use a variety of ionizations techniques such as electrospray ionization (ESI), matrix-assisted laser desorption ionization (MALDI), surface-enhanced laser desorption ionization (SELDI), fast-atom bombardment (FAB), chemical ionization (CI), electron impact (EI), inductively coupled plasma (ICP), field desorption ionization (FDI), atmospheric pressure chemical ionization (APCI), or any derivative of these techniques.

In another aspect, the invention provides that multiple biomolecules in the same sample may be measured simultaneously, or multiplexed. That is, the amount of unlabeled biomolecules and their respective Quantitation Standards may be detected and measured separately or at the same time in the same extract fraction for multiple biomolecules of interest. As such, the invention provides a useful method for screening changes in concentration of one or more biomolecules on a large scale (i.e., proteomics/metabolomics) and provides a sensitive means to detect and quantify biomolecules involved in the underlying pathophysiology. In this aspect, the invention also provides a means to measure multiple types of biomolecules. In this context, for example, a protein and a lipid may be measured simultaneously or sequentially within the same or different phases (liquid or pellet) of the extraction solvents.

Once the amount of unlabeled biomolecule(s) has been detected in a sample, the concentration of unlabeled biomolecule(s) may be determined. In other words, since a known amount of Quantitation Standard is added to an unknown amount of biomolecules and the ratio of unlabeled to Quantitation Standard is measured, the concentration of the unlabeled biomolecules can be calculated from the ratio as follows:

Concentration of unlabeled=(unlabeled signal intensity:Quantitation Standard signal intensity)×(known concentration of Quantitation Standard). (i)

In another embodiment, the methods further include the step of normalizing the calculated concentration to a standard curve based on the curve fitting equation generated by the standard curve. The standard curve used herein is generated by determining two or more ratios of unlabeled biomolecules to their respective Quantitation Standards, where the concentration of the unlabeled biomolecule of interest is known.

However, prior to detecting the ratio of unlabeled biomolecule to the Quantitation Standard, it may be desirable to isolate and/or separate the biomolecule of interest from other biomolecules in the sample. In one aspect, Aβ isoforms are isolated from the biologic samples by precipitation. Those of skill in the art will appreciate that several precipitation reagents can be used and are not limited to organic solvents, organic acids, bases, inorganic salts, and heat. In this embodiment, the precipitated proteins (“pellet”) are solubilized in acidic conditions, physiologically neutral, or basic conditions and then digested with cyanogen bromide producing Aβ isoform specific C-terminal fragments (CTFs). Those of skill in the art will appreciate that several solubilization reagents can be used and are not limited to organic solvents, organic acids, and bases. A liquid-liquid extraction is performed using an organic solvent to extract the highly hydrophobic Aβ CTFs. The invention allows one to measure the concentration of Aβ isoforms (e.g., Aβ40 and Aβ42) in complex matrices such as plasma and CSF.

In an exemplary embodiment, the digestion agent can be a chemical cleavage reagent (e.g., CNBr, hydroxylamine, acids, BNPS-skatole, 2-nitro-5-thiocyanobenzoic acid, o-iodosobenzoic acid). In the current context, this digestion strategy will produce Aβ isoform specific CTFs.

In another embodiment, the digestion agent can be, but is not limited to, commonly used endoproteases (e.g., trypsin, Lys-C, Lys-N, Glu-C, LysArgiNase, Neprolysin, insulin degrading enzyme, MMP2, MMP9, MMP14, cathepsins, plasmin, or V8 protease) or a combination thereof. Appropriate endoproteases could also include other aspartic acid, cysteine, glutamic acid, metalloproteases, serine, and threonine proteases as listed in Table 3.

In yet another embodiment, the digestion reagent can be a synthetic palladium (Pd) or platinum (Pt) metal peptidase compound (Ni and Kanai, 2016) which produces Aβ isoform specific CTFs. Those of skill in the art can appreciate that the metal ion complexes used to target specific amino acid side chains for peptide cleavage could be, but are not limited to Pd, Pt, Zn, Co, Zr, Cu, Ni, and Mo.

In an exemplary embodiment, the biomolecule (e.g., peptide or protein) of interest may be extracted with an organic solvent of varying polarity and then detected, identified, characterized, and quantified using a liquid chromatography system interfaced with a tandem MS unit equipped with an electrospray ionization source (LC-ESI-tandem MS).

The term “organic solvent” as used in this invention is meant to include any carbon-based, low molecular weight, lipophilic compound or mixture of compounds, capable of dissolving or extracting a biomolecule of interest for analysis.

In yet another embodiment, the biomolecule of interest may be extracted using an aqueous two phase system (ATPS) consisting of the combination of two or more polymers (eg. polyethylene glycol or dextran), salts (eg. phosphate, citrate, or sulfate), ionic liquids, short chain alcohols, or ionic/nonionic surfactants (Iqbal et al., Biological Procedures Online, 2016).

When detecting the ratio of unlabeled biomolecule to the Quantitation Standard, it may be desirable to isolate and/or separate the biomolecule of interest from other biomolecules in the sample. Thus, in one embodiment, the biomolecule(s) may be precipitated to isolate the analyte of interest. Alternatively, sample introduction may be through a chromatographic inlet (e.g. reverse phase, cation exchange, anion exchange, HILIC, or ERLIC) used to separate complex mixtures of biomolecules without using immunoprecipitation prior to mass spectrometry detection. This allows for direct separation, detection, identification, characterization, and quantification of the biomolecule(s) of interest present in a complex matrix. In one embodiment, this strategy utilizes a liquid chromatography system interfaced with a tandem MS unit equipped with an electrospray ionization source (LC-ESI-tandem MS).

In another embodiment, immunoprecipitation may be used to isolate and purify the biomolecule (e.g., peptide or protein) of interest before it is analyzed. The immunocapture may be performed with an antibody raised to the protein (intact or a proteolytic fragment) prior to digestion. In another embodiment, the biomolecule of interest may be isolated or purified by affinity chromatography or immunoaffinity chromatography. In yet another embodiment, the immunocapture may be performed with an antibody raised to a proteolytic or chemically digested peptide from the protein target of interest. In another embodiment, an antibody or collection of more than one antibody may be used to capture and deplete unwanted or interfering biomolecules from a sample, and enrich for the biomolecules of interest in the remaining sample.

The term “antibody” as used in this invention is meant to include intact molecules of polyclonal or monoclonal antibodies, as well as fragments thereof, such as Fab and F(ab′)₂, Fv and SCA fragments which are capable of binding an epitopic determinant present on the biomolecule of interest. The term “specifically binds” or “specifically interacts,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less.

In one aspect, this methodology can be applied to stable isotope kinetic labeling (SILK) in living subjects. SILK measurements have been shown to detect in vivo metabolic incorporation of stable (non-radioactive) isotopes into newly synthesized proteins in the cerebrospinal fluid, plasma, and tissues of living subjects. For detailed information regarding SILK, see U.S. Pub. Nos. 2008/0145941 and 2009/0142766, and International PCT Pub. No. WO 2006/107814, the entire content of each of which is incorporated herein by reference). SILK makes it possible to measure the in vivo production and clearance rates of proteins in the central nervous system.

Combined with stable isotope labeling kinetics (SILK) for measuring the ratio of labeled biomolecules at different time points after administration of a labeled moiety, the methodology presented herein allows for the calculation of absolute concentration of newly synthesized biomolecules (e.g., peptides or proteins) and/or the absolute concentration of each of the isoforms of that biomolecule.

The method of the invention may be used to diagnose or monitor the progression of a neurological, neurodegenerative, or pathophysiological disease by measuring the in vivo concentration of one or more biomolecules of interest in a subject. Additionally, the methods of the invention may be used to monitor the treatment of a neurological or neurodegenerative disease by measuring the in vivo concentration of a biomolecule of interest in a subject. The concentration of the biomolecule may be linked to any disease such that any increase or decrease in biomolecule concentration may be indicative of the absence, presence, progression, or mitigation of the disease. Thus, the calculated concentration of one or more biomolecules of interest may be compared to the concentration of the same biomolecules in a corresponding normal sample, to the concentration of the same biomolecules in a subject with a known disease state (or states), to the concentration of the same biomolecules from the same subject determined at an earlier time, or any combination thereof.

In addition, such methods may help identify an individual as having a predisposition for the development of a disease, or may provide a means for detecting the disease prior to the appearance of actual clinical symptoms. A more definitive diagnosis of this type may allow health professionals to employ preventative measures or aggressive treatment earlier, thereby preventing the development or further progression of the disease.

As used herein a “corresponding normal sample” refers to a sample from the same organ and/or of the same tissue or biofluid type as the sample being examined. In one aspect, the corresponding normal sample comprises a sample of cells obtained from a healthy subject. Such a corresponding normal sample can, but need not be, from a subject that is age-matched and/or of the same sex as the subject providing the sample being examined. In another aspect, the corresponding normal sample comprises a sample of cells obtained from an otherwise healthy portion of tissue of the subject from which the sample being tested is obtained.

Reference to the concentration of biomolecules in a subject of known disease state(s) includes a predetermined concentration of a biomolecule linked to a disease. Thus, the concentration may be compared to a known concentration of biomolecules obtained from a sample of a single subject or may be from an established cell line of the same type as that of the “diseased” subject. In one aspect, the established cell line can be one of a panel of such cell lines, wherein the panel can include different cell lines of the same type of disease and/or different cell lines of different diseases associated with the same biomolecule. Such a panel of cell lines can be useful, for example, to practice the present method when only a small number of cells can be obtained from the subject to be treated, thus providing a surrogate sample of the subject's cells, and also can be useful to include as control samples in practicing the present methods.

Exemplary diseases that may be linked to the concentration ranges of biomolecules of interest include, but are not limited to, Alzheimer's Disease, Pick's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases (e.g., Creutzfeldt-Jakob Disease, bovine spongiform encephalopathy or Mad Cow Disease, and scrapie), Lewy Body Disease, schizophrenia, Amyotrophic Lateral Sclerosis (ALS or Lou Gehrig's Disease) or other motor neuron diseases, restless legs syndrome, epilepsy or other seizure disorders, tremors, depression, mania, anxiety disorders, brain trauma or injury, narcolepsy, insomnia or other sleep disorders, autism, normal pressure hydrocephalus, pain disorders or syndromes, migraines, cluster headaches or other forms of headache, spinocerebellar disorders, muscular dystrophies, myasthenia gravis, retinitis pigmentosa or other forms of retinal degeneration. It is also envisioned that the method of the invention may be used to study the normal physiology, metabolism, and function of a subject without disease.

In another aspect, the present invention provides a method for assessing whether a therapeutic agent used to treat a disease affects the concentration of a biomolecule of interest in the subject. For example, the concentration of the biomolecule may be measured to determine if a given therapeutic agent results in an increase, or a decrease in the concentration of the biomolecule. In one embodiment, the method is performed in vivo, as herein described. In another embodiment, the method is performed in vitro utilizing a culture of cells, where the culture of cells is the “subject” in the methods described herein. Accordingly, use of the methods provided herein will allow those of skill in the art to accurately determine the degree of change in the concentration of the biomolecule of interest, and correlate these measurements with the clinical outcome of the disease modifying treatment. Results from this aspect of the invention, therefore, may help determine the optimal doses and frequency of doses of a therapeutic agent, may assist in the decision-making regarding the design of clinical trials, and may ultimately accelerate validation of effective therapeutic agents for the treatment of disease(s).

Thus, the method of the invention may be used to predict which subjects will respond to a particular therapeutic agent. For example, subjects with increased concentrations of a particular biomolecule may respond to a particular therapeutic agent differently than subjects with decreased concentrations of the biomolecule. In particular, results from the method may be used to select the appropriate treatment (e.g., an agent that blocks the production of the biomolecule or an agent that increases the clearance of the biomolecule) for a particular subject. Similarly, results from the method may be used to select the appropriate treatment for a subject having a particular genotype.

Those of skill in the art will appreciate that the therapeutic agent can and will vary depending upon the disease or disorder to be treated and/or the biomolecule(s) being detected, identified, characterized, and quantified. For example, in embodiments in which the biomolecule is Aβ, non-limiting examples of suitable therapeutic agents include Aβ metabolism modulators, Aβ kinase inhibitors, cathepsin inhibitors, and Aβ aggregation inhibitors. Other suitable AD therapeutic agents include hormones, neuroprotective agents, and cell death inhibitors. Many of the above mentioned therapeutic agents may also affect the in vivo metabolism and concentration of other biomolecules implicated in diseases and disorders, and the methods in this invention can be applied to these other biomolecules. Furthermore, therapeutic agents that may affect the in vivo metabolism of α-synuclein include sirtuin 2 inhibitors, synuclein aggregation inhibitors, proteasome inhibitors, etc.

The therapeutic agent may be administered to the subject in accordance with known methods. Typically, the therapeutic agent will be administered orally, but other routes of administration such as parenteral or topical may also be used. The amount of therapeutic agent that is administered to the subject can and will vary depending upon the type of agent, the subject, and the particular mode of administration. Those skilled in the art will appreciate that dosages may be determined with guidance from Goodman & Goldman's The Pharmacological Basis of Therapeutics, Tenth Edition (2001), Appendix II, pp. 475-493, and the Physicians' Desk Reference.

It should be understood that the methods of the invention described herein can be adapted to a high throughput format, thus allowing the examination of a plurality (i.e., 2, 3, 4, or more) of samples and/or biomolecules, which independently can be the same or different, in parallel. A high throughput format provides numerous advantages. For example, a high throughput format allows for the detection, identification, characterization, examination, and quantitation of two, three, four, etc., different biomolecules, alone or in combination, in a subject and sample. Finally, a high throughput format allows, for example, control samples (positive controls and or negative controls) to be run in parallel with test samples. In addition a high throughput method may allow extraction of multiple proteins at the same time allowing for measurement of multiple different biomolecules in a single sample.

The following examples are provided to further illustrate the advantages and features of the present invention, but are not intended to limit the scope of the invention. While they are typical of those that might be used, other procedures, methodologies, detectors, or techniques known to those skilled in the art may alternatively be used.

### Example 1

### Quantitation of Endogenous Aβ by SISAQ

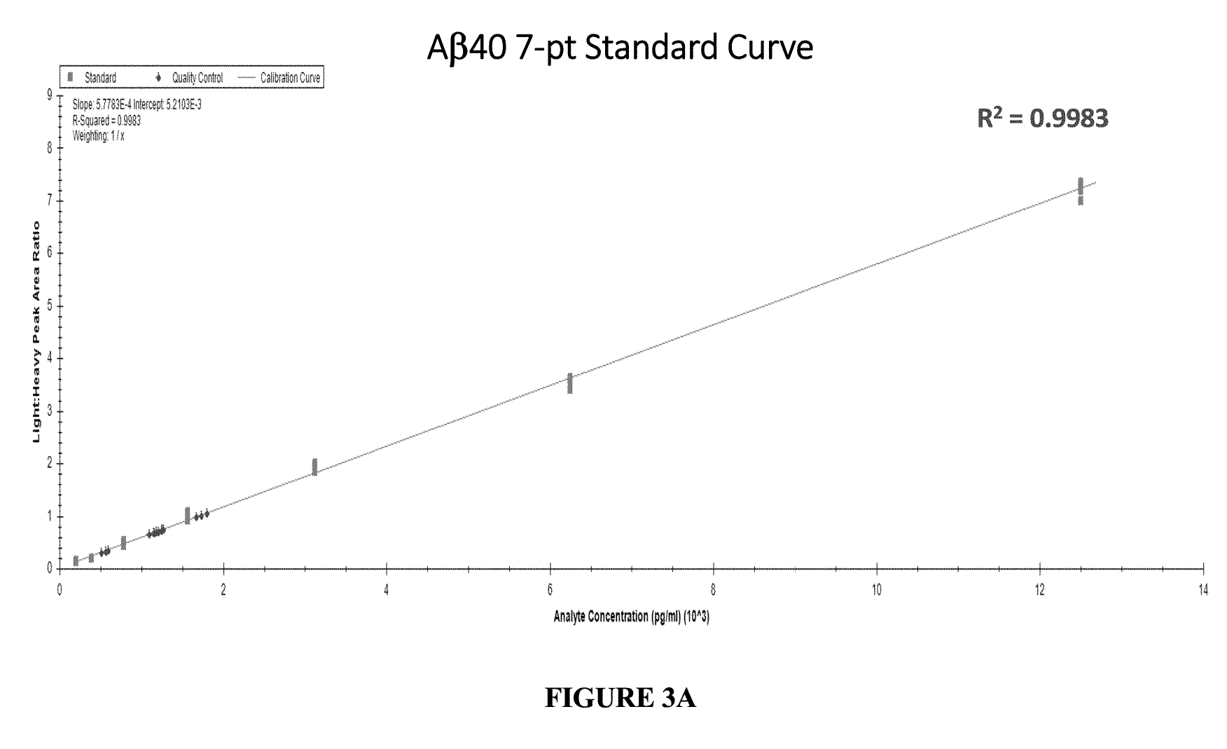

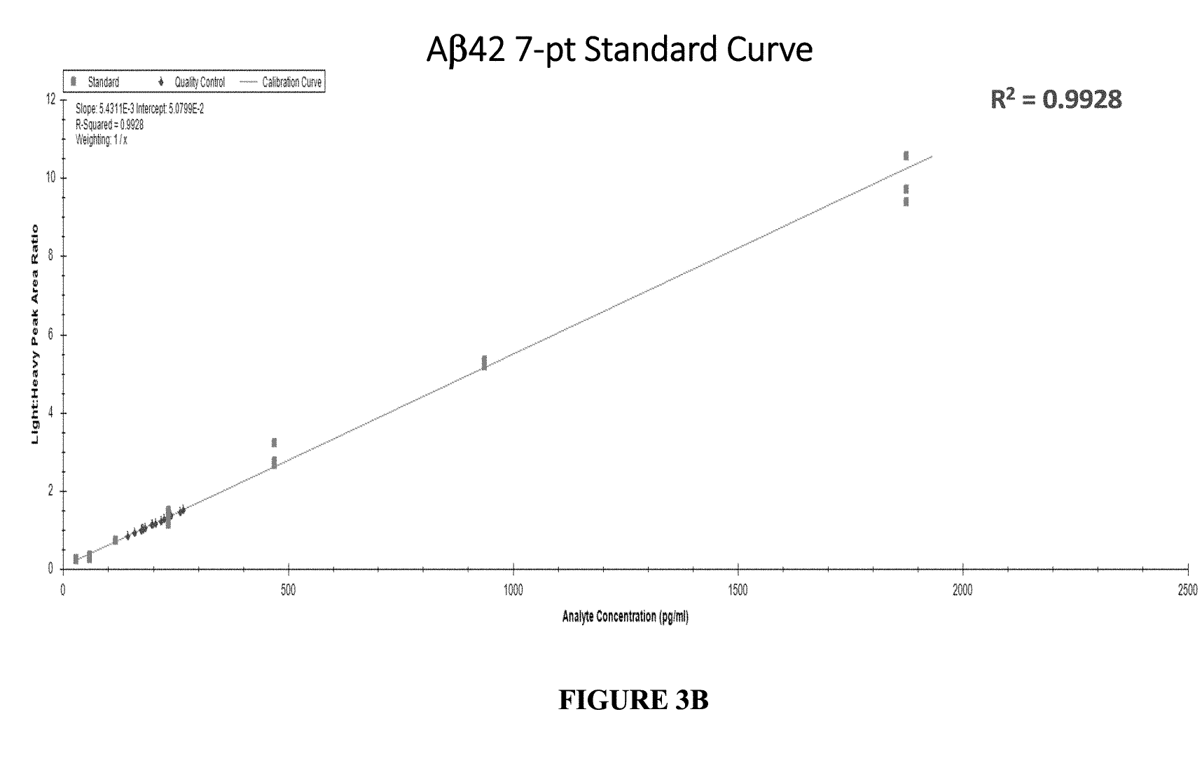

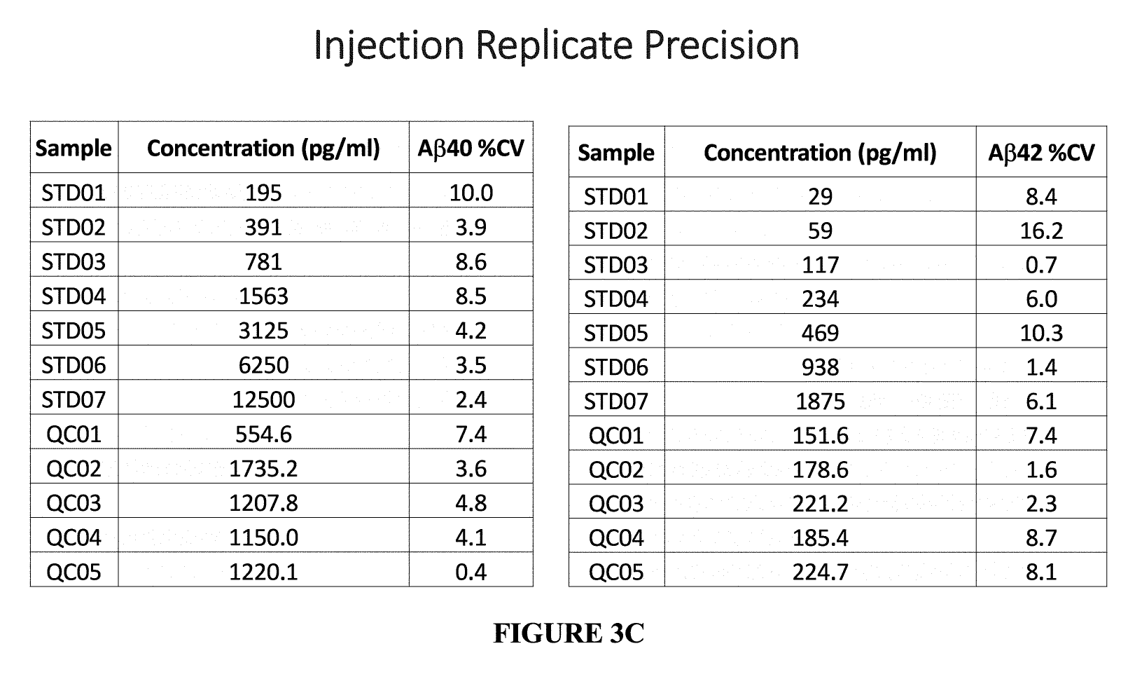

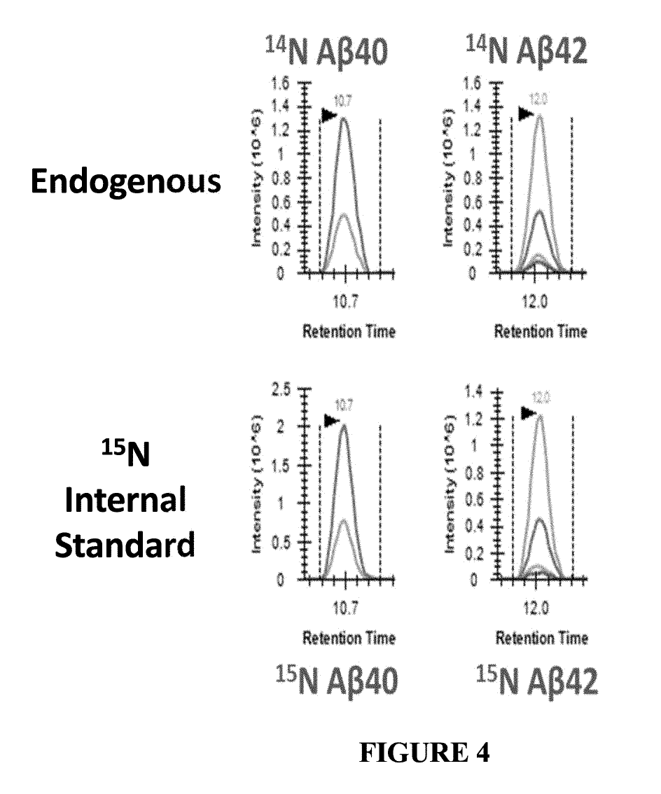

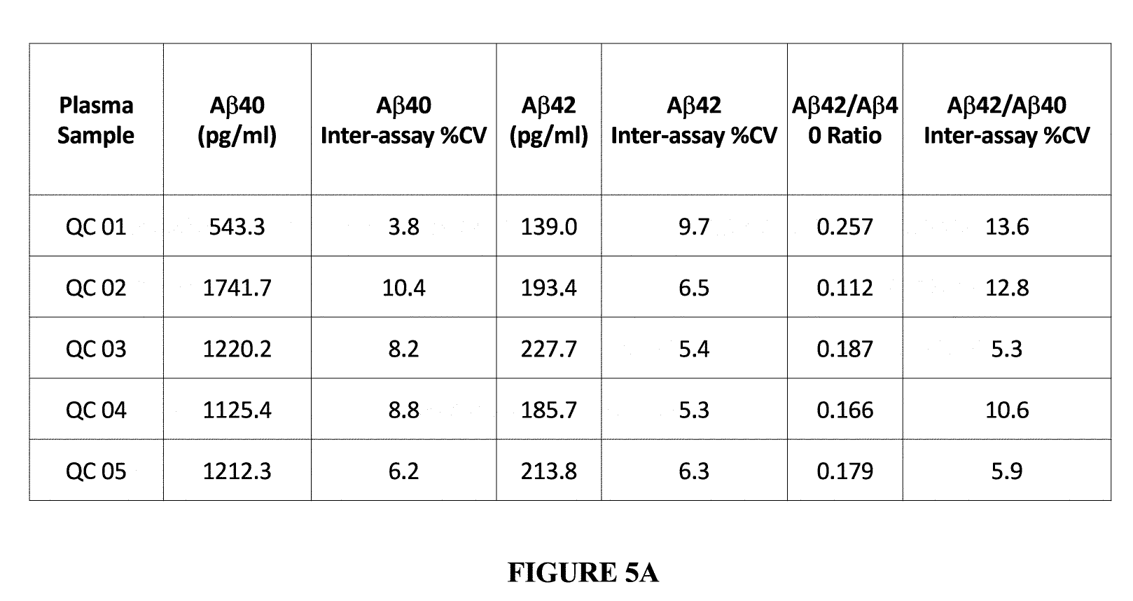

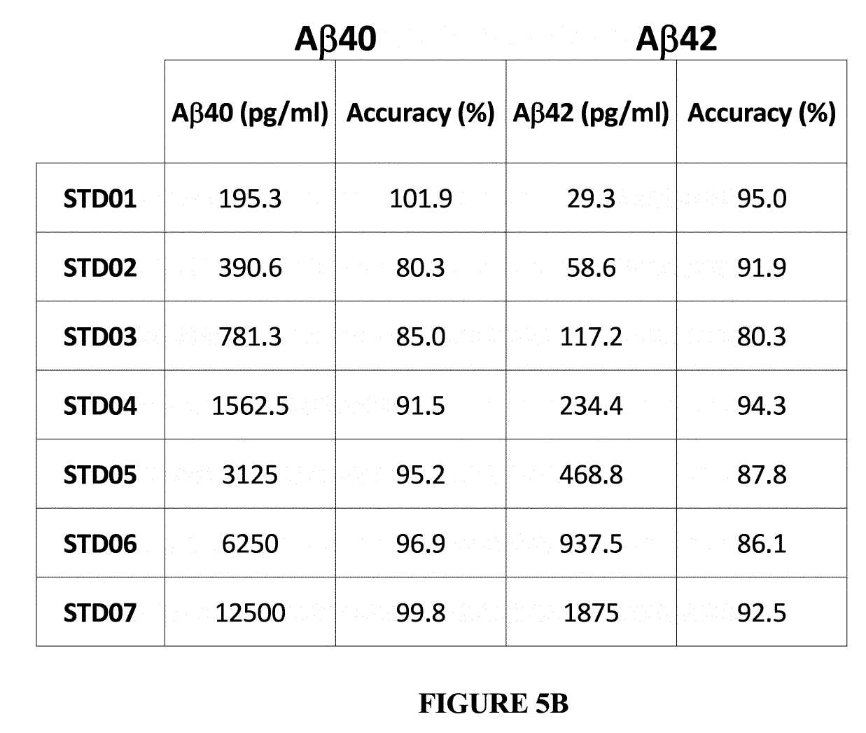

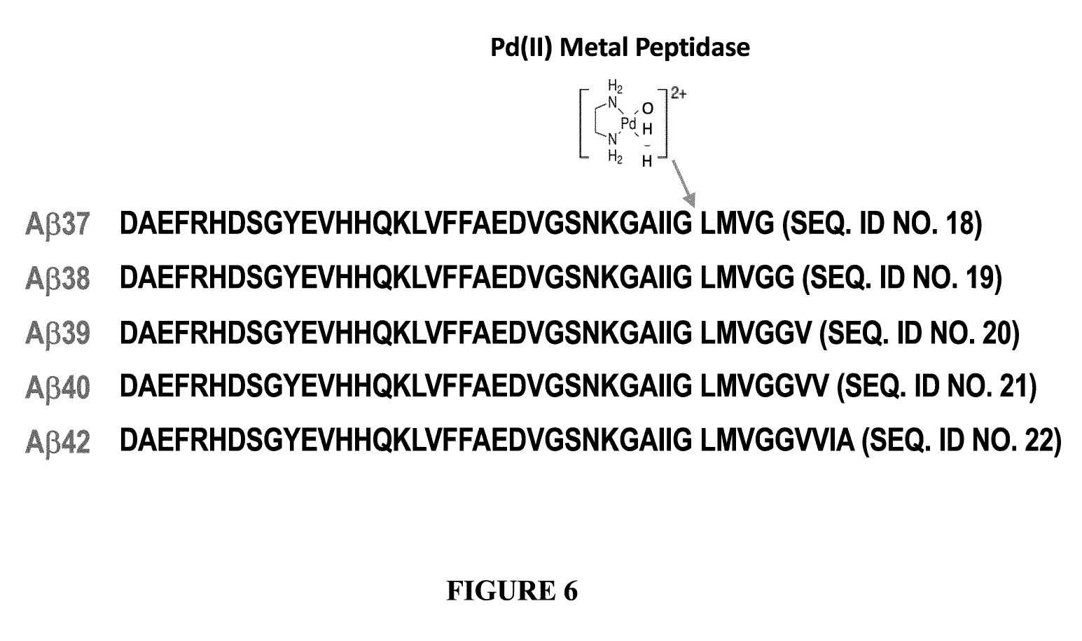

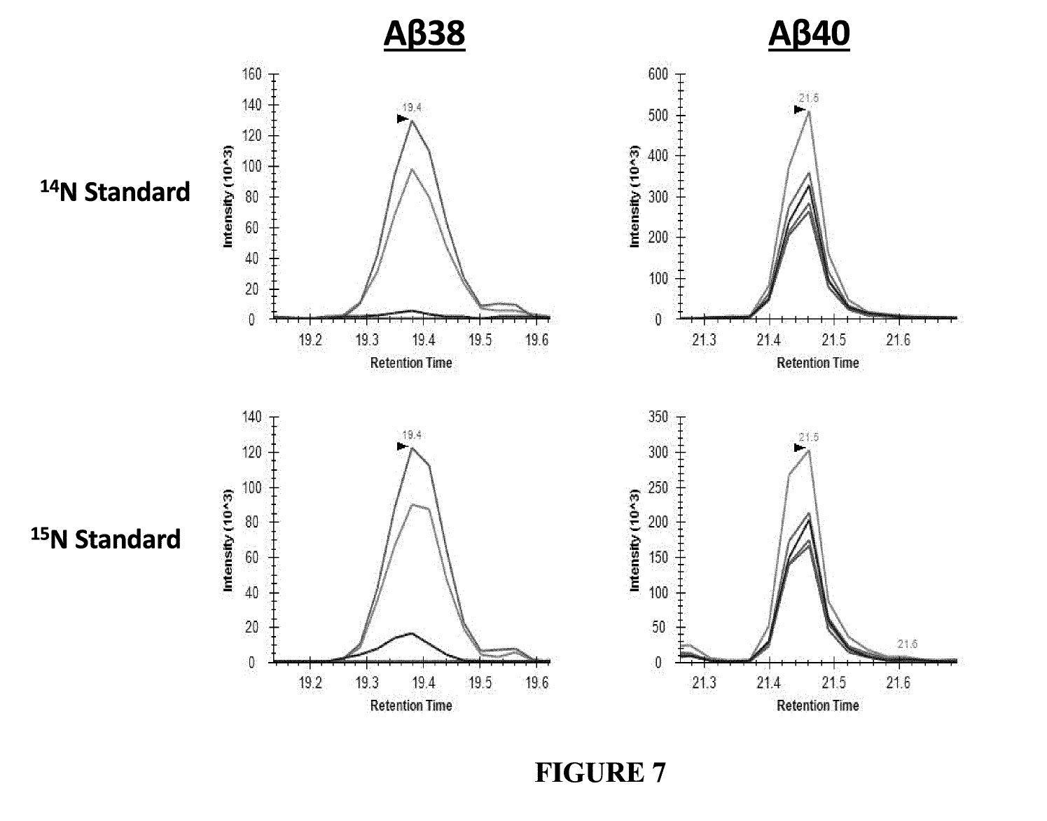

Five separate human plasma QC samples were spiked with uniformly labeled ¹⁵N Aβ40/Aβ42 used as Quantitation Standard at concentrations of 1667 pg/ml and 250 pg/ml, respectively (FIG. 5A). The Aβ isoforms were extracted from the QC samples using precipitation, digested with cyanogen bromide, and the Aβ CTFs were obtained by liquid-liquid extraction. The resulting extracts were dried down, peptides were cleaned up by solid phase extraction, and then analyzed by liquid chromatography-mass spectrometry (LC-MS). The top two and the top four most abundant fragment ions were monitored for the Aβ40 and Aβ42 CTFs, respectively. The amount of unlabeled, endogenous Aβ relative to the known amount of corresponding Quantitation Standard (ratio of their summed fragment ion signal intensities) was calculated for all the samples and were quantified using 7-point calibration curves for the Aβ40 and Aβ42 CTFs (FIGS. 3A and 3B). The concentrations in plasma range from 554.6-1735.2 pg/mL for Aβ40 and from 151.6-224.7 pg/mL for Aβ42 and the precision (% CV) of triplicate measurements for both calibrators and samples was <20% (FIG. 3C).

To demonstrate the reproducibility of the methods, a set of five QC plasma samples and a 7-pt standard curve (described above) was processed on five separate days and each QC and calibrator set was analyzed by LC-MS. The Aβ isoforms were digested, extracted, and analyzed as described above. The amount of unlabeled, endogenous Aβ relative to the known amount of corresponding Quantitation Standard (ratio of their summed fragment ion signal intensities) and were quantified using 7-point calibration curves for the Aβ40 and Aβ42 CTFs. The inter-assay variation (% CV) for the Aβ40 and Aβ42 concentration measurements and the Aβ42/Aβ40 concentration ratio were <15%, indicating a robust and reproducible assay (FIG. 5A). In addition, a spike recovery experiment was performed to evaluate plasma matrix effects on Aβ recovery. Pooled plasma samples were spiked with the same concentrations used in the 7-point calibration curve and were compared to an unspiked plasma sample. The amount of Aβ40 and Aβ42 spiked at each level is shown in FIG. 5B. The accuracy of the measurements was between 80-120% for all concentrations of Aβ40 and Aβ42 tested (FIG. 5B).

This illustrates the feasibility of using this novel workflow for the isolation of endogenous Aβ and a stable isotope labeled Aβ Quantitation Standard, and using the relative ratio of unlabeled to a labeled Aβ Quantitation Standard signal intensities to calculate the concentrations of the Aβ isoforms in biological samples.

### Example 2

### Identification of Plasma Proteins by CNBr Digestion and Liquid-Liquid Extraction

To demonstrate the utility of the invention and its application for identifying and quantifying other biomolecules in addition to Aβ, 500 μL aliquots of human plasma were prepared as described herein, and analyzed using liquid chromatography-mass spectrometry. Briefly, plasma proteins were precipitated, resolubilized, and digested with cyanogen bromide. The digestion reactions underwent liquid-liquid extraction with a series of organic solvents varying in polarity. Those skilled in the art will recognize that many different organic solvents, or combinations thereof, can be used in this extraction step, and the identity of the organic solvent(s) will be determined by the type of analyzer the user selects for detecting, identifying, characterizing, and quantifying the biomolecule(s) of interest. In this example, extracted fractions were dried down and analyzed by high-resolution LC-MS/MS operating in data-dependent mode. The mass spectra data were searched and matched against annotated libraries that contain theoretical mass spectra for human proteins. Using this sample extraction method and LC-MS/MS approach, a list of 298 proteins were identified, wherein 228 identified proteins had high confidence protein identification scores (protein false-discovery rate (FDRs)<0.05). This illustrates that this method can be used to isolate and quantify many biomolecules (i.e., human plasma proteins) in addition to Aβ isoforms that may be associated with disease.

The present invention is further illustrated in Exhibits A and B, attached hereto and incorporated by reference in their entirety.

Although the invention has been described with reference to the above examples, it will be understood that modifications and variations are encompassed within the spirit and scope of the invention. Accordingly, the invention is limited only by the following claims.

1. A method of calculating the concentration of biomolecules in a subject comprising:
(a) contacting a sample from the subject with Quantitation Standards, wherein the Quantitation Standards comprise known concentrations of labeled or tagged biomolecules of interest;
(b) extracting the biomolecules of interest from the sample; and
(c) calculating the concentrations of the unlabeled biomolecules in the sample.

2. The method of claim 1, wherein calculating the concentration of the unlabeled biomolecule comprises multiplying the known concentration of the Quantitation Standard with the ratio of unlabeled biomolecule to the Quantitation Standard in the sample.

3. The method of claim 1, further comprising normalizing the calculated concentration to a standard curve, wherein the standard curve is generated by determining two or more ratios of unlabeled biomolecule to the Quantitation Standard, wherein the concentration of the unlabeled biomolecule is known.

4. The method of claim 1, wherein the Quantitation Standard comprises one or more labeled moieties.

5. The method of claim 4, wherein the one or more labeled moieties comprise a non-radioactive isotope that is selected from the group consisting of 2H, 13C, 15N, 17O, 18O, 33S, 34S, 36S, 74Se, 76Se, 77Se, 78Se, and 80Se.

6. The method of claim 1, wherein the biomolecule is selected from the group consisting of proteins/peptides, lipids, nucleic acids, metabolites, and carbohydrates.

7. The method of claim 6, wherein the biomolecule is a protein that is present, synthesized, or secreted from the cells of a subject.

8. The method of claim 7, wherein the peptide is a protein selected from the group consisting of tau, amyloid-beta (Aβ), alpha-synuclein, apolipoprotein E, apolipoprotein J, amyloid precursor protein (APP), alpha-2-macroglobulin, S100B, myelin basic protein, TDP-43, superoxide dismutase-1, huntingtin, an interleukin, TNF, and the proteins listed in Table 2.

9. The method of claim 4, wherein the labeled moiety is an amino acid.

10. The method of claim 9, wherein the amino acid is an essential amino acid.

11. The method of claim 10, wherein the essential amino acid is selected from the group consisting of histidine, tryptophan, threonine, glutamic acid, leucine, isoleucine, valine, methionine, and phenylalanine.

12. The method of claim 4, wherein the labeled moiety is 13Cx-threonine, where x=1 to 4.

13. The method of claim 4, wherein the labeled moiety is 13Cx-glutamic acid, where x=1 to 5.

14. The method of claim 4, wherein the peptide is Aβ and the Quantitation Standard contains a 13Cx-glycine, where x=1 to 2.

15. The method of claim 4, wherein the peptide is Aβ and the Quantitation Standard contains a 13Cx-alanine, where x=1 to 3.

16. The method of claim 4, wherein the labeled moiety is 13Cx-leucine, where x=1 to 6.

17. The method of claim 4, wherein the labeled moiety is 13Cx-valine, where x=1 to 5.

18. The method of claim 4, wherein the labeled moiety is 13Cx-methionine, where x=1 to 5.

19. The method of claim 4, wherein the labeled moiety is 33, 34, 35S-methionine.

20. The method of claim 4, wherein the labeled moiety is 74, 76, 77, 78, 80Se-methionine.

21. The method of claim 4, wherein the labeled moiety is a 13Cx-labeled isoleucine, where x=1 to 6.

22. The method of claim 4, wherein the labeled moiety is a 13Cx-labeled lysine, where x=1 to 6.

23. The method of claim 4, wherein the labeled moiety is a 15N-labeled amino acid.

24. The method of claim 1, wherein the sample is contacted with two or more Quantitation Standards and the concentrations of two or more unlabeled biomolecules are calculated.

25. The method of claim 24, wherein the two or more biomolecules are isoforms of the same protein.

26. The method of claim 24, wherein the biomolecules are selected from the group consisting of Aβ 1-16, Aβ 1-17, Aβ 1-37, Aβ 1-38, Aβ 1-39, Aβ 1-40, Aβ 1-41, Aβ 1-42, and Aβ 1-43.

27. The method of claim 26, wherein the biomolecules are selected from the group consisting of post-translationally modified Aβ.

28. The method of claim 1, wherein the sample is selected from the group consisting of cerebral spinal fluid (CSF), blood, blood cells, blood serum, blood plasma, urine, saliva, breath condensate, perspiration (sweat), tears, vitreous fluid, and biological tissue.

29. The method of claim 28, wherein the sample is CSF and comprises (among other biomolecules) tau, amyloid-beta protein, variants of amyloid-beta protein, digestion products of amyloid-beta protein, amyloid precursor protein (APP), apolipoprotein E, apolipoprotein J, alpha-synuclein, alpha-2-macroglobulin, S100B, myelin basic protein, TDP-43, superoxide dismutase-1, huntingtin, an interleukin, TNF, the proteins listed in Table 2, or any combination thereof.

30. The method of claim 1, wherein the unlabeled biomolecule in the sample is detected by a means selected from the group consisting of mass spectrometry, tandem mass spectrometry, and a combination thereof.

31. The method of claim 29, wherein the biomolecule is a peptide derived from a protein digested with a proteolytic reagent prior to determining the concentration.

32. The method of claim 31, wherein the proteolytic reagent is cyanogen bromide, hydroxylamine, acids, BNPS-skatole, 2-nitro-5-thiocyanobenzoic acid, or o-iodosobenzoic acid.

33. The method of claim 31, wherein the proteolytic reagent is a synthetic Pd or Pt metal peptidase complex.

34. The method of claim 31, wherein the proteolytic reagent is an endoprotease comprising of trypsin, LysN, Glu-C, LysArgiNase, Neprolysin, insulin degrading enzyme, MMP2, MMP9, MMP14, cathepsins, plasmin, or V8 protease. Appropriate endoproteases could also include other aspartic acid, cysteine, glutamic acid, metalloproteases, serine, and threonine proteases as listed in Table 3.

35. The method of claim 31, wherein the digested peptide is separated by chromatographic separation before the concentration is determined.

36. The method of claim 35, wherein the chromatographic separation comprises liquid or gas chromatography.

37. The method of claim 1, wherein the biomolecule is separated from complex background matrix by precipitation with a chemical reagent.

38. The method of claim 37, wherein the chemical reagent is an organic solvent, organic acid, base, or inorganic salt.

39. The method of claim 1, wherein the biomolecule is further extracted by liquid-liquid extraction with an organic solvent.

40. The method of claim 39, wherein the organic solvent is methyl tert-butyl ether, methanol, ethanol, isopropanol, chloroform, ethyl acetate, trichloroethylene, dichloromethane, xylenes, hexane, or any solvent contained in the GMP Q3C Solvent Tables and List (FDA Guidance, ICH, 2012, revision 2).

41. The method of claim 39, wherein the organic solvent is a combination of two or more organic solvents consisting of methyl tert-butyl ether, methanol, ethanol, isopropanol, chloroform, ethyl acetate, trichloroethylene, dichloromethane, xylenes, hexane, or any solvent contained in the GMP Q3C Solvent Tables and List (FDA Guidance, ICH, 2012, revision 2).

42. The method of claim 1, wherein the biomolecule is further extracted using a liquid-liquid aqueous two-phase system (ATPS).

43. The method of claim 42, the biomolecule of interest may be extracted using an ATPS consisting of the combination of two or more polymers (eg. polyethylene glycol or dextran), salts (eg. phosphate, citrate, or sulfate), ionic liquids, short chain alcohols, or ionic/nonionic surfactants (Iqbal et al., Biological Procedures Online, 2016).

44. The method of claim 1, further comprising comparing the concentration of the unlabeled biomolecule of interest to the concentration of the same biomolecule in a corresponding normal sample, to the concentration of the same biomolecule in a subject of known disease state, to the concentration of the same biomolecule from the same subject determined at an earlier time, or any combination thereof.

45. The method of claim 44 wherein the disease is selected from the group consisting of Alzheimer's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases, Lewy Body Disease, Amyotrophic Lateral Sclerosis, as well as other pathophysiological diseases.

46. An extraction and analytical method for quantifying the concentration of one or more biomolecules in a subject comprising:
(a) obtaining a sample of biological fluid or tissue from the subject, wherein the sample contains an unlabeled biomolecule fraction;
(b) contacting the sample with a Quantitation Standard, wherein the Quantitation Standards comprise known concentrations of biomolecules, each labeled with a moiety that has a molecular weight or a chemical “tag” that allows the skilled user to select an analyzer capable of differentiating and quantifying one or more unlabeled biomolecule(s) from the Quantitation Standard(s);
(c) determining a ratio of the signal intensity for the unlabeled biomolecule(s) to the signal intensity for the Quantitation Standard(s); and
(d) calculating the concentrations of the unlabeled biomolecules in the sample.

47.-90. (canceled)

91. A method of calculating the concentrations of biomolecules in a subject comprising:
(a) Extracting the biomolecules of interest from the sample;
(b) Contacting the sample extract with Quantitation Standards, wherein the Quantitation Standards comprise known concentrations of labeled or tagged biomolecules of interest;
(c) Calculating the concentrations of the unlabeled biomolecules in the sample extract.


SEQUENCE LISTING (22 sequences)

SEQ ID NO: 1 - protein, 36 aa, Homo sapiens
  misc_feature: Amyloid beta 1-36
DAEFRHDSGYEVHHQKLVFFAEDVGSNKGAIIGLMV

SEQ ID NO: 2 - protein, 36 aa, Homo sapiens
  misc_feature: Amyloid beta 1-37
AEFRHDSGYEVHHQKLVFFAEDVGSNKGAIIGLMVG

SEQ ID NO: 3 - protein, 38 aa, Homo sapiens
  misc_feature: Amyloid beta- 138
DAEFRHDSGYEVHHQKLVFFAEDVGSNKGAIIGLMVGG

SEQ ID NO: 4 - protein, 39 aa, Homo sapiens
  misc_feature: Amyloid beta 1-39
DAEFRHDSGYEVHHQKLVFFAEDVGSNKGAIIGLMVGGV

SEQ ID NO: 5 - protein, 40 aa, Homo sapiens
  misc_feature: Amyloid beta 1-40
DAEFRHDSGYEVHHQKLVFFAEDVGSNKGAIIGLMVGGVV

SEQ ID NO: 6 - protein, 41 aa, Homo sapiens
  misc_feature: Amyloid beta 1-41
DAEFRHDSGYEVHHQKLVFFAEDVGSNKGAIIGLMVGGVVI

SEQ ID NO: 7 - protein, 42 aa, Homo sapiens [sequence omitted]

SEQ ID NO: 8 - protein, 43 aa, Homo sapiens
  misc_feature: Amyloid beta 1-43
DAEFRHDSGYEVHHQKLVFFAEDVGSNKGAIIGLMVGGVVIAT

SEQ ID NO: 9 - protein, 5 aa, Homo sapiens
  misc_feature: Amyloid beta 40
VGGVV

SEQ ID NO: 10 - protein, 6 aa, Homo sapiens
  misc_feature: Amyloid beta 40
NVGGVV

SEQ ID NO: 11 - protein, 7 aa, Homo sapiens
  misc_feature: Amyloid beta 42
VGGVVIA

SEQ ID NO: 12 - protein, 8 aa, Homo sapiens
  misc_feature: Amyloid beta 42
NVGGVVIA

SEQ ID NO: 13 - protein, 37 aa, Homo sapiens
  misc_feature: Amyloid beta 37
DAEFRHDSGYEVHHQKLVFFAEDVGSNKGAIIGLMVG

SEQ ID NO: 14 - protein, 38 aa, Homo sapiens; identical to SEQ ID NO: 3
  misc_feature: Amyloid 38

SEQ ID NO: 15 - protein, 39 aa, Homo sapiens; identical to SEQ ID NO: 4
  misc_feature: Amyloid beta 39

SEQ ID NO: 16 - protein, 40 aa, Homo sapiens; identical to SEQ ID NO: 5
  misc_feature: Amyloid beta 40

SEQ ID NO: 17 - protein, 42 aa, Homo sapiens [sequence omitted]

SEQ ID NO: 18 - protein, 37 aa, Homo sapiens; identical to SEQ ID NO: 13
  misc_feature: Amyloid beta 37

SEQ ID NO: 19 - protein, 38 aa, Homo sapiens; identical to SEQ ID NO: 3
  misc_feature: Amyloid beta 38

SEQ ID NO: 20 - protein, 39 aa, Homo sapiens; identical to SEQ ID NO: 4
  misc_feature: Amyloid beta 39

SEQ ID NO: 21 - protein, 40 aa, Homo sapiens; identical to SEQ ID NO: 5
  misc_feature: Amyloid beta 40

SEQ ID NO: 22 - protein, 42 aa, Homo sapiens [sequence omitted]